(19) United States
(12) Patent Application Publication
(10) Pub. No.: US 2023/0282335 A1
(43) Pub. Date: Sep. 7, 2023

(54) COLLATION APPARATUS, COLLATION METHOD, AND COLLATION PROGRAM

(71) Applicants: Hitachi, Ltd., Tokyo (JP); NATIONAL UNIVERSITY CORPORATION HOKKAIDO UNIVERSITY, Sapporo-shi (JP)

(72) Inventors: Miaomei LEI, Tokyo (JP); Takahiro NAKAMURA, Tokyo (JP); Takashi TAKEMOTO, Tokyo (JP); Michinori MAYAMA, Sapporo-shi (JP)

(21) Appl. No.: 18/101,418

(22) Filed: Jan. 25, 2023

(30) Foreign Application Priority Data
Feb. 2, 2022 (JP) 2022-015123

(51) Int. Cl. G16H 30/40

(52) U.S. Cl. CPC G16H 30/40

(57) ABSTRACT

An object of the invention is to improve reliability of an annotation. A collation apparatus includes: a grouping processing unit configured to group, based on each explanatory variable of a sample group, the sample group into a first group indicating a first classification and a second group indicating a second classification having a lower evaluation than the first classification; a collation unit configured to collate the classification of the first group and the second group that are obtained by the grouping processing unit with a classification identified by each objective variable of the sample group; and an output unit configured to output a collation result obtained by the collation unit.

### CLAIM OF PRIORITY

The present application claims priority from Japanese patent application JP 2022-015123 filed on Feb. 2, 2022, the content of which is hereby incorporated by reference into this application.

### BACKGROUND OF THE INVENTION

### 1. Field of the Invention

The present invention relates to a collation apparatus, a collation method, and a collation program for collating data.

### 2. Description of the Related Art

Cardio toco gram (CTG) is a waveform showing temporal changes in fetal heart rate and a tocogram (uterine contraction) obtained from a fetal heart rate monitor and an external tocodynamometer, respectively, and is used to evaluate well-being of a fetus. The CTG is an essential test for evaluating the fetus during delivery. The CTG is useful for early detection of low oxygen and acidosis of the fetus that may occur during delivery, and for reducing hypoxic encephalopathy and cerebral palsy in an unborn fetus.

A doctor performs an evaluation of the CTG according to a level classification in which an evaluation is executed based on a baseline of a fetal heart rate and a pattern of bradycardia. However, the waveform of the CTG is complicated and depends on experiences of a medical worker who makes a determination. Ramanujam, E., et al. “Prediction of Fetal Distress Using Linear and Non-linear Features of CTG Signals.” International Conference On Computational Vision and Bio Inspired Computing. Springer, Cham, 2019 (Non-Patent Literature 1) discloses a technique for predicting a fetal state based on a fetal heart rate signal.

However, a prediction method presented in Non-Patent Literature 1 described above is a technique of predicting a fetal state by extracting feature data from waveforms of the fetal heart rate and the uterine contraction using annotated data of a doctor, and the method requires well-organized annotated data. Since an annotation of data depends on experiences of the doctor, there is a variation in reliability. In addition, the annotation performed by the doctor can be only classified into several categories (for example, safety or danger), and a pH value cannot be predicted according to the annotation alone.

### SUMMARY OF THE INVENTION

An object of the invention is to improve reliability of an annotation.

A collation apparatus according to an aspect of the invention disclosed in the present application includes: a grouping processing unit configured to group, based on each explanatory variable of a sample group, the sample group into a first group indicating a first classification and a second group indicating a second classification having a lower evaluation than the first classification; a collation unit configured to collate the classification of the first group and the second group that are obtained by the grouping processing unit with a classification identified by each objective variable of the sample group; and an output unit configured to output a collation result obtained by the collation unit.

According to a representative embodiment of the invention, reliability of an annotation is improved. Problems, configurations, and effects other than those described above are made clear according to the following description of the embodiments.

### BRIEF DESCRIPTION OF THE DRAWINGS

FIG. 1 is an explanatory diagram showing an example of well-organization of a learning DB.

FIG. 2 is a block diagram of a hardware configuration example of a collation apparatus.

FIG. 3 is an explanatory diagram showing an example of the learning DB.

FIG. 4 is an explanatory diagram showing a measuring factor example 1 included in feature data.

FIG. 5 is an explanatory diagram showing a measuring factor example 2 included in the feature data.

FIG. 6 is an explanatory diagram showing an example of a basic information DB.

FIG. 7 is a block diagram showing a functional configuration example of the collation apparatus.

FIG. 8 is a flowchart showing an example of a preprocessing procedure performed by a preprocessing unit.

FIG. 9 is an explanatory diagram showing a display example of a selection screen during learning.

FIG. 10 is an explanatory diagram showing a display example of a selection screen during prediction.

FIG. 11 is a flowchart showing an example of a collation processing procedure during learning.

FIG. 12 is an explanatory diagram showing an example of a feature data selection screen.

FIG. 13 is an explanatory diagram showing an example of a collation result display screen.

FIG. 14 is an explanatory diagram showing an example of the collation result display screen.

FIG. 15 is an explanatory diagram showing an example of the collation result display screen.

FIG. 16 is an explanatory diagram showing an example of the collation result display screen.

FIG. 17 is a flowchart showing an example of a collation processing procedure during prediction.

### DESCRIPTION OF THE PREFERRED EMBODIMENTS

### Example of Well-Organization of Learning DB

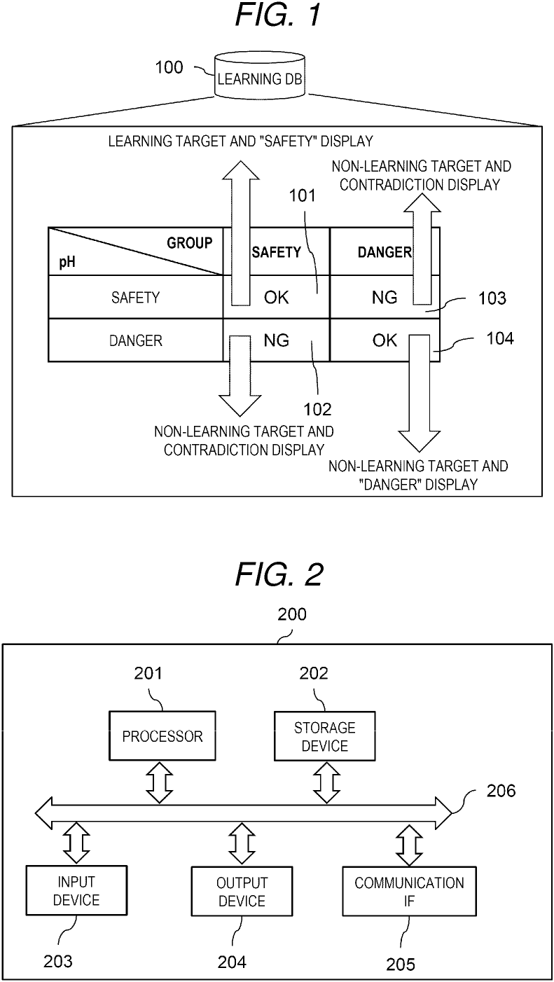

FIG. 1 is an explanatory diagram showing an example of well-organization of a learning DB. A learning DB 100 is a database that stores, as sample data, a combination of feature data serving as an explanatory variable and correct data serving as an objective variable for each sample which is a pregnant woman. The correct data is, for example, an annotation (a fetal state is safe or dangerous) performed by a doctor for a delivered sample during learning, and is an annotation (the fetal state is safe or dangerous) obtained during learning for a pre-delivery sample during pH prediction.

Each piece of sample data is clustered into a safety group in which the fetal state is safe and a danger group in which the fetal state is dangerous. Clustering is executed in unsupervised learning that does not use the correct data or supervised learning that uses the correct data. Here, the unsupervised learning that does not use the correct data will be described as an example. In the unsupervised learning that does not use the correct data, a sample data group is clustered into two groups, in which a group having a larger number of pieces of sample data is set as the safety group and a group having a smaller number of pieces of sample data is set as the danger group.

pH is an umbilical arterial blood gas analysis value immediately after delivery serving as an index of low oxygen or acidosis of a fetus. For example, a pH value is set to 7.2 as a reference. When the pH > 7.2, the fetus is safe, and when the pH ≤ 7.2, the fetus is dangerous. The pH value is an actual measurement value immediately after delivery in the case of the delivered sample, and is a predicted value before delivery in the case of the pre-delivery sample.

If a collation result 101 indicates that the sample data belongs to the safety group and the pH value is a safety value (both safe), the sample data is stored in the learning DB 100 as a learning target, and an annotation indicating “safety” is attached to the sample data. The annotation indicating “safety” is displayed to a user.

If a collation result 102 indicates that the sample data belongs to the safety group and the pH value is a danger value (contradiction), the sample data is set as a non-learning target in the learning DB 100 so as not to be used for learning, and a case indicating that an attribute “safety” of the sample and the safety value of the pH are “contradictory” is displayed to a user.

If a collation result 103 indicates that the sample data belongs to the danger group and the pH value is the safety value (contradiction), the sample data is set as the non-learning target in the learning DB 100 so as not to be used for learning, and a case indicating that an attribute “danger” of the sample and the safety value of the pH are “contradictory” is displayed to a user.

If a collation result 104 indicates that the sample data belongs to the danger group and the pH value is the danger value (both dangerous), the sample data is set as the non-learning target in the learning DB 100 so as not to be used for learning, and an annotation indicating “danger” is attached to the sample data. The annotation indicating “danger” is displayed to a user.

In this way, the collation results 101 to 104 indicating “safety”, “contradiction”, and “danger” are automatically attached as annotations. Accordingly, reliability of the annotations can be improved. In addition, since the sample data to which the collation results 102 to 104 indicating “contradiction” or “danger” are attached as annotations serves as the non-learning target, the sample data to which the collation result 101 indicating “safety” is attached as an annotation remains. Therefore, automatic organization of the learning DB 100 can be implemented. In addition, by generating a learning model using the remaining sample data group, accuracy of a predicted value of the pH can be improved.

### Hardware Configuration Example of Collation Apparatus

FIG. 2 is a block diagram of a hardware configuration example of a collation apparatus. A collation apparatus 200 includes a processor 201, a storage device 202, an input device 203, an output device 204, and a communication interface (communication IF) 205. The processor 201, the storage device 202, the input device 203, the output device 204, and the communication IF 205 are connected by a bus 206. The processor 201 controls the collation apparatus 200. The storage device 202 serves as a work area of the processor 201. The storage device 202 is a non-transitory or transitory recording medium that stores various programs and data. Examples of the storage device 202 include a read only memory (ROM), a random access memory (RAM), a hard disk drive (HDD), and a flash memory. The input device 203 inputs data. Examples of the input device 203 include a keyboard, a mouse, a touch panel, a numeric keypad, a scanner, a microphone, and a sensor. The output device 204 outputs data. Examples of the output device 204 include a display, a printer, and a speaker. The communication IF 205 is connected to a network and transmits and receives the data.

### Learning DB 100

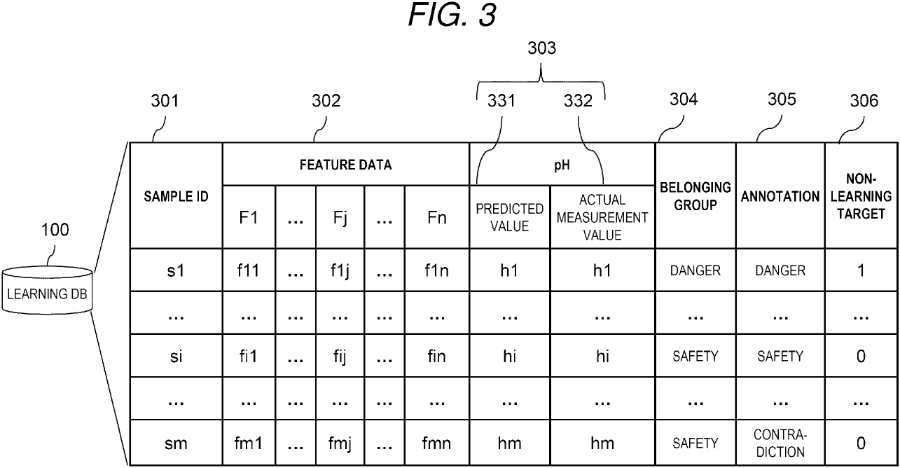

FIG. 3 is an explanatory diagram showing an example of the learning DB 100. The learning DB 100 is stored in the storage device 202 or a storage device of another computer accessible to the collation apparatus.

The learning DB 100 includes, as fields, sample IDs 301, feature data 302, pHs 303, belonging groups 304, annotations 305, and non-learning target information 306. A combination of values of the fields 301 to 306 in the same row defines an entry indicating sample data of one sample. Although the sample represents a pregnant woman, when a heart rate and labor intensity of the same pregnant woman are measured at different dates and times, samples will be different. In FIG. 3, sample data of m sample or m samples (m is an integer of 1 or more) is registered.

Each of the sample IDs 301 is identification information that uniquely identifies a sample. The feature data 302 is data indicating features of the sample identified by each of the sample IDs 301. The feature data 302 includes factors F1 to Fn (n is an integer of 1 or more) including basic factors such as age, the number of weeks of pregnancy, development delay of a fetus, the number of fetuses, a delivery method, medication information, and smoking for a pregnant woman, and measurement factors such as measurement results obtained by a measurement apparatus that measures a heart rate and labor intensity.

The pHs 303 are the umbilical arterial blood gas analysis values immediately after delivery, and predicted values 331 and actual measurement values 332 are stored therein. Before delivery, only the predicted values 331 are calculated and stored by a calculation unit 703 to be described later. After delivery, the umbilical arterial blood gas analysis values are measured and stored as the actual measurement values 332.

Each of the belonging groups 304 is a group to which the sample identified by the sample ID 301 belongs. The group includes the safety group and the danger group. As described with reference to FIG. 1, when clustering is performed by the unsupervised learning, the group having a larger number of pieces of sample data is set as the safety group and the group having a smaller number of pieces of sample data is set as the danger group.

On the other hand, the belonging groups 304 of the sample data may be determined by using a pH value = 7.2 as a reference for the actual measurement values 332, attaching a correct label indicating safety when the actual measurement values 332 are pH > 7.2, attaching a correct label indicating danger when the actual measurement values 332 are pH ≤ 7.2, and executing the supervised learning.

Each of the annotations 305 is an evaluation index for a fetus of a sample, and is attached by a doctor or attached as a collation result by a collation unit 704 to be described later.

The non-learning-target information 306 is information for setting the sample data to be the non-learning target. “0” indicates a default value of the learning target, and “1” indicates the non-learning target. It is noted that the collation apparatus may delete an entry of sample data serving as the non-learning target, without using the non-learning target information 306.

### Feature Data 302

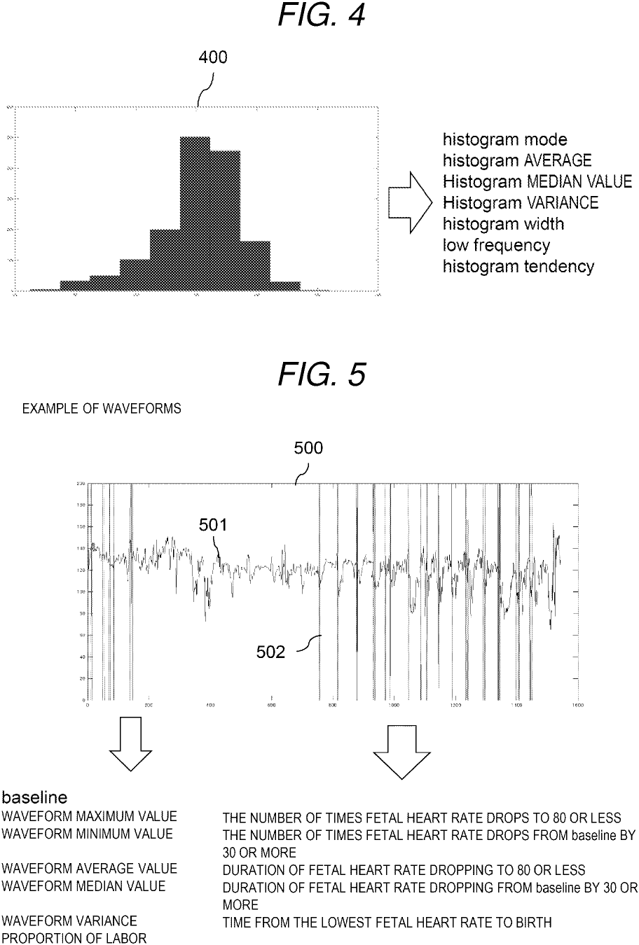

FIG. 4 is an explanatory diagram showing a measuring factor example 1 included in the feature data 302. FIG. 4 shows a histogram 400 obtained based on waveforms of CTG. Factors of the feature data 302 obtained based on the histogram 400 include, for example, a mode, an average value, a median value, variance, a histogram width, a low frequency, and a tendency.

FIG. 5 is an explanatory diagram showing a measuring factor example 2 included in the feature data 302. FIG. 5 shows waveforms of CTG. The waveforms include a heart rate waveform 501 and a labor intensity waveform 502. Factors obtained based on the heart rate waveform 501 include, for example, a baseline, a waveform maximum value, a waveform minimum value, a waveform average value, and waveform variance. Factors obtained based on the labor intensity waveform 502 include, for example, a proportion of labor (total labor time/measurement period). In addition, factors obtained as medical knowledge include, for example, the number of times fetal heart rate drops to 80 [bpm] or less, the number of times the fetal heart rate drops from the baseline by 30 [bpm] or more, duration of the fetal heart rate dropping to 80 [bpm] or less, duration of the fetal heart rate dropping from the baseline by 30 [bpm] or more, and time from the lowest fetal heart rate to birth.

### Basic Information DB

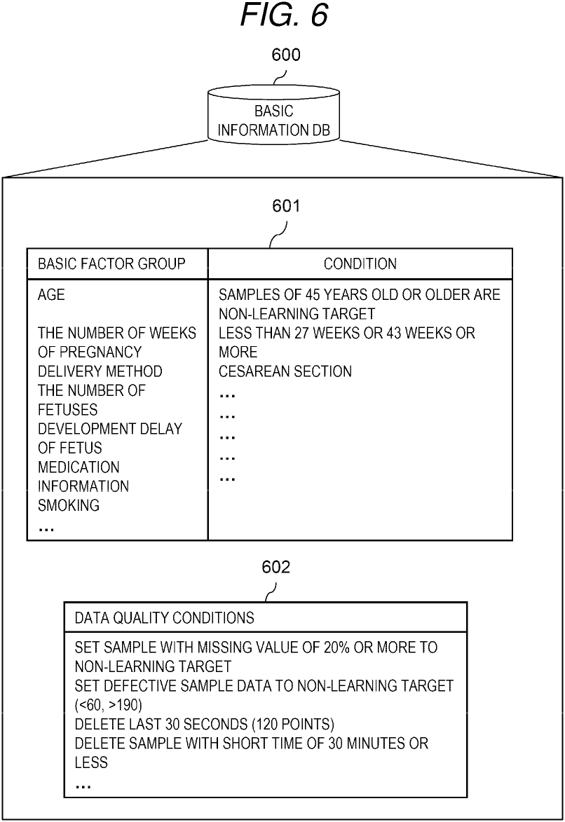

FIG. 6 is an explanatory diagram showing an example of a basic information DB. The basic information DB 600 is stored in the storage device 202 or a storage device of another computer accessible to the collation apparatus.

The basic information DB 600 stores data used for cleansing the sample data. Specifically, for example, the basic information DB 600 includes conditions 601 related to a basic factor group and data quality conditions 602. In the conditions 601 related to the basic factor group, for example, the basic factor group such as the age, the number of weeks of pregnancy, the delivery method, the number of fetuses, the development delay of the fetus, the medication information, and the smoking for a pregnant woman who is a sample serves as the non-learning target. For example, in the case of age, if sample data is 45 years old or older, the sample data is set to the non-learning target. In addition, in the case of the number of weeks of pregnancy, sample data of less than 27 weeks or 43 weeks or more is set to the non-learning target. In this way, a condition serving as the non-learning target is set for each factor.

The data quality conditions 602 are conditions related to quality of values of sample data, which are different from the conditions 601 related to the basic factor group. For example, sample data with a missing value of 20% or more is set to the non-learning target. It is noted that the non-learning target may be, as long as the sample data is not used for learning, a state in which the sample data itself is simply left in the learning DB 100 or a state in which the sample data is deleted from the learning DB 100. It is noted that even if the sample data is deleted from the learning DB 100, the sample data may remain in the storage device 202.

### Functional Configuration Example of Collation Apparatus

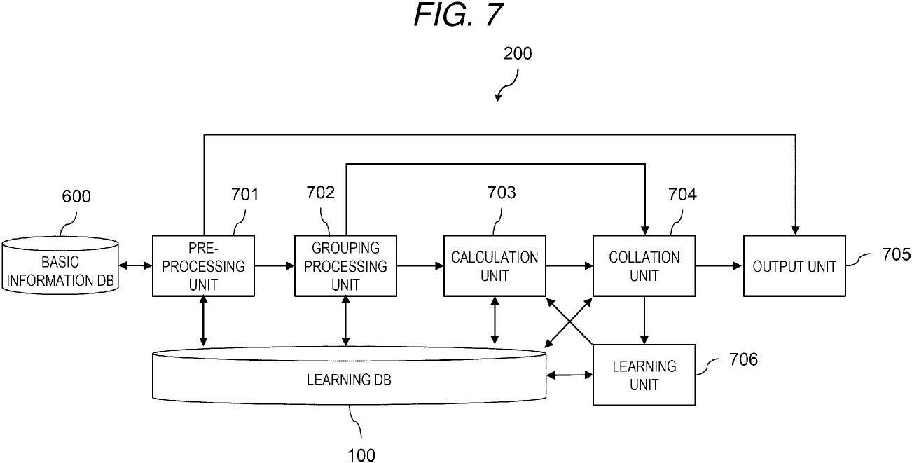

FIG. 7 is a block diagram showing a functional configuration example of the collation apparatus 200. The collation apparatus 200 can access the learning DB 100 and the basic information DB 600. The collation apparatus 200 includes a preprocessing unit 701, a grouping processing unit 702, the calculation unit 703, the collation unit 704, an output unit 705, and a learning unit 706. Specifically, the preprocessing unit 701, the grouping processing unit 702, the calculation unit 703, the collation unit 704, the output unit 705, and the learning unit 706 are implemented by, for example, causing the processor 201 to execute a program stored in the storage device 202 shown in FIG. 2.

The preprocessing unit 701 refers to the basic information DB 600, cleanses the sample data group in the learning DB 100, and sets unnecessary sample data to the non-learning target.

The grouping processing unit 702 groups the sample data group in the learning DB 100 into the safety group and the danger group. Specifically, for example, as described above, the grouping processing unit 702 executes, by the unsupervised learning, grouping between the sample data having short distances between the feature data 302 (specifically, for example, a measurement factor group) of the sample data, and executes grouping until the sample data finally converge into two groups. The final two groups are the safety group and the danger group. The group having a larger number of pieces of sample data is the safety group, and the group having a smaller number of pieces of sample data is the danger group.

The grouping processing unit 702 classifies a combination of the feature data 302 (specifically, the measurement factor group) and a correct label (safety if pH > 7.2, and danger if pH ≤ 7.2) based on the actual measurement values 332 of the pHs 303 as training data into the safety group and the danger group by the supervised learning. Since the actual measurement values 332 of the pHs 303 are used, sample data to be grouped serves as sample data of a delivered pregnant woman.

The calculation unit 703 calculates the predicted values 331 of the pHs 303 by inputting a specific measurement factor group (a factor group obtained as medical knowledge) of sample data (prediction target sample data) for which the predicted values 331 of the pHs 303 have not been calculated to a learning model generated by the learning unit 706.

As shown in FIG. 1, the collation unit 704 collates the annotations 305 with a safety value (pH > 7.2) or a danger value (pH ≤ 7.2) obtained based on the pHs 303 for each sample data. As for the pHs 303, the actual measurement values 332 are used for sample data having the actual measurement values 332, and the predicted values 331 are used for sample data having no actual measurement values 332.

Each of the annotations 305 is an annotation performed by a doctor before the collation executed by the collation unit 704, and becomes a collation result obtained by the collation unit 704 after the collation executed by the collation unit 704.

The output unit 705 outputs a preprocessing result obtained by the preprocessing unit 701 and the collation result obtained by the collation unit 704 in a displayable manner. Specifically, for example, the output unit 705 displays the preprocessing result or the collation result on a display apparatus which is an example of the output device 204, or transmits the preprocessing result or the collation result to another computer via the communication IF 205 to display the preprocessing result or the collation result on the other computer.

The learning unit 706 generates a multiple regression model as the learning model using the factor group obtained as medical knowledge of the sample data in the feature data 302 and the actual measurement values 332 of the pHs 303.

### Collation Processing Procedure

Next, an example of a collation processing procedure performed by the collation apparatus 200 will be described for each function. The collation apparatus 200 executes learning of the learning model and prediction of the pHs 303 using the learning model, and will be described by indicating which of learning and prediction the collation apparatus 200 is applied to in processes performed by functions to be described later.

### Example of Preprocessing Procedure

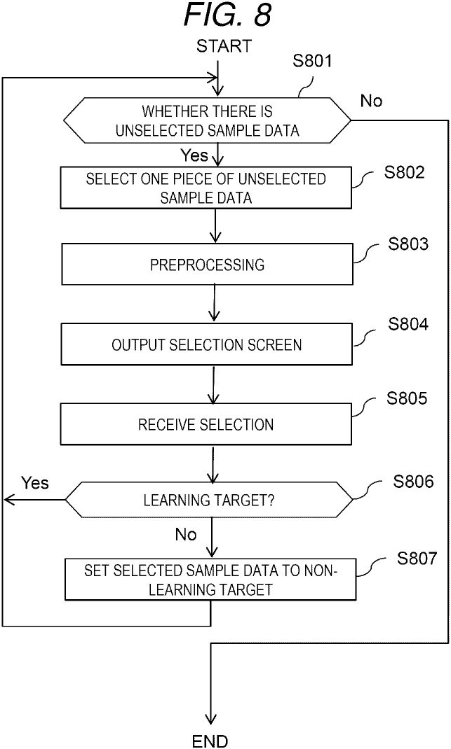

FIG. 8 is a flowchart showing an example of a preprocessing procedure performed by the preprocessing unit 701. The preprocessing procedure is performed in both learning and prediction. The preprocessing unit 701 determines whether there is unselected sample data in the learning DB 100 (step S801). When there is the unselected sample data (step S801: Yes), the preprocessing unit 701 selects one piece of unselected sample data (step S802). The preprocessing unit 701 executes preprocessing (cleansing), that is, a process of determining whether selected sample data corresponds to the conditions 601 related to the basic factor group and the data quality conditions 602, on the selected sample data, using the basic information DB 600 (step S803).

Then, the preprocessing unit 701 outputs a selection screen in the displayable manner (step S804). Specifically, for example, the preprocessing unit 701 executes displaying on a display apparatus, which is an example of the output device 204, or executes displaying on another computer operated by a user.

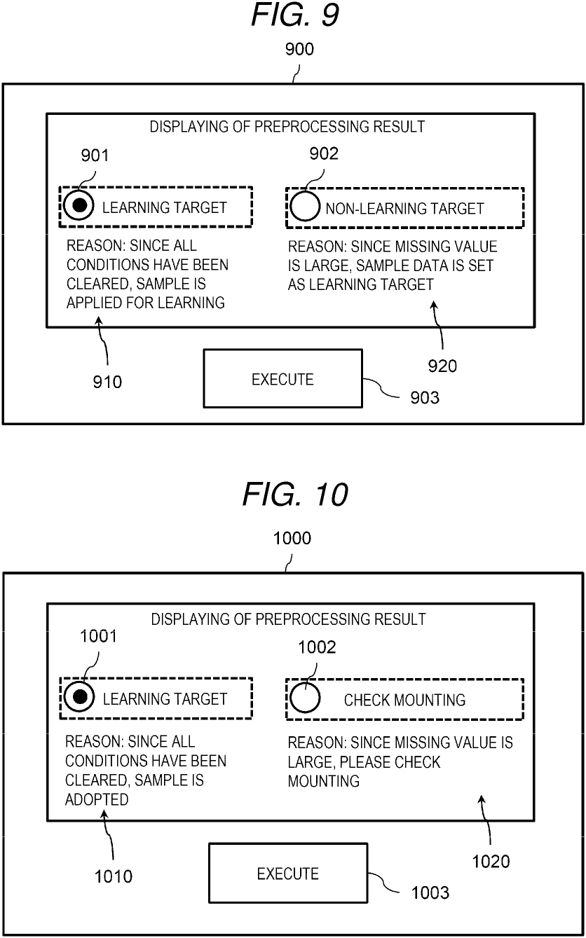

FIG. 9 is an explanatory diagram showing a display example of a selection screen during learning. A selection screen 900 displays, as a preprocessing result, a first radio button 901, a second radio button 902, an execution button 903, a first character string 910, and a second character string 920.

The first radio button 901 is a selection button for a user to set the preprocessed selected sample data to the learning target, and a reason thereof is displayed as the first character string 910. “All conditions” of the first character string 910 are the conditions 601 related to the basic factor group and the data quality conditions 602 in the basic information DB 600. That is, it is indicated that the selected sample data is the learning target.

The second radio button 902 is a selection button for a user to set the preprocessed selected sample data to the non-learning target, and a reason thereof is displayed as the second character string 920. The second character string 920 is, for example, a condition corresponding to the selected sample data among the conditions 601 related to the basic factor group and the data quality conditions 602 in the basic information DB 600.

The execution button 903 is a button for checking selection of either the first radio button 901 or the second radio button 902, and the preprocessing unit 701 receives a signal indicating whether the selected sample data is selected as the learning target or the non-learning target.

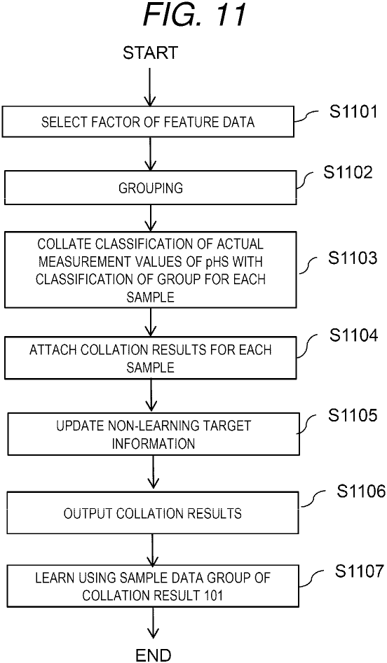

FIG. 10 is an explanatory diagram showing a display example of a selection screen during prediction. A selection screen 1000 displays, as a preprocessing result, a first radio button 1001, a second radio button 1002, an execution button 1003, a first character string 1010, and a second character string 1020.

The first radio button 1001 is a selection button for a user to set the preprocessed selected sample data to the learning target, and a reason thereof is displayed as the first character string 1010. “All conditions” of the first character string 1010 are the conditions 601 related to the basic factor group and the data quality conditions 602 in the basic information DB 600. That is, it is indicated that the selected sample data is the learning target.

The second radio button 1002 is a selection button for a user to set the preprocessed selected sample data to the non-learning target and for prompting the user to recheck mounting of the measurement apparatus, and content for prompting the rechecking is displayed as the second character string 1020. The execution button 1003 is a button for checking selection of either the first radio button 1001 or the second radio button 1002, and the preprocessing unit 701 receives a signal indicating whether the selected sample data is selected as the learning target or the non-learning target.

Referring back to FIG. 8, when the execution button 903 or the execution button 1003 is pressed, the preprocessing unit 701 receives a selection signal (step S805). When the selection signal indicates the learning target (step S806: Yes), the process returns to step S801. On the other hand, when the selection signal indicates the non-learning target (step S806: No), the preprocessing unit 701 sets the non-learning target information 306 of the selected sample data to the non-learning target (step S807), and returns to step S801. In step S801, when there is no unselected sample data, the preprocessing unit 701 ends the preprocessing.

### Example of Collation Processing Procedure During Learning

FIG. 11 is a flowchart showing an example of a collation processing procedure during learning. The grouping processing unit 702 selects a factor of the feature data (step S1101). Specifically, for example, the grouping processing unit 702 outputs a feature data selection screen at start of collation in the displayable manner.

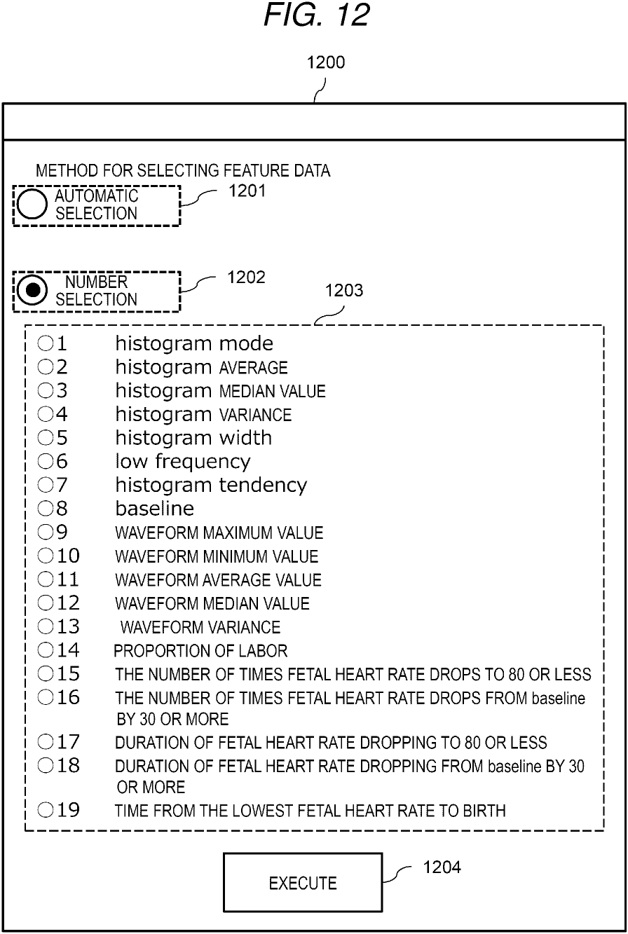

FIG. 12 is an explanatory diagram showing an example of the feature data selection screen. A feature data selection screen 1200 includes an automatic selection button 1201, a number selection button 1202, a factor group list 1203, and an execution button 1204. The automatic selection button 1201 is a button for the collation apparatus 200 to automatically select a factor of the feature data 302. The number selection button 1202 is a button for selecting any one or more factors from a factor group in the factor group list 1203. A user selects a radio button (indicated by a circle) of each factor at a left end of the factor group list 1203. When the execution button 1204 is pressed, the grouping processing unit 702 receives a selection method signal indicating automatic selection or number selection (including a selection number).

Referring back to FIG. 11, the grouping processing unit 702 selects the factor of the feature data when receiving the selection method signal. Specifically, for example, in the case of the automatic selection, the grouping processing unit 702 automatically selects a factor of the feature data 302 according to T-test or principal component analysis. In the case of the number selection, the grouping processing unit 702 selects a number-selected factor.

The grouping processing unit 702 executes a grouping process on the sample data group in the learning DB 100 using the factor selected in step S1101 as the feature data 302 (step S1102). Sample data to be subjected to the grouping process (step S1102) is sample data having the actual measurement values 332 of the pHs 303 and having the non-learning target information 306 of “0” (learning target). Accordingly, since defective data is not applied to the grouping process (step S1102), accuracy of the grouping process (step S1102) during learning is improved.

During learning, in the grouping process (step S1102), the grouping processing unit 702 may execute the supervised learning using selection factors of the sample data and correct data (a correct label obtained based on the actual measurement values 332 of the pHs 303) as a learning data set, or may execute the unsupervised learning using only the selection factors of the sample data. In any case, the grouping processing unit 702 groups the sample data group into the safety group and the danger group, and sets the belonging groups 304.

Next, as shown in FIG. 1, the collation unit 704 collates a classification (safety or danger) of the actual measurement values 332 of the pHs 303 with a classification (safety or danger) of the belonging groups 304 for each piece of sample data (step S1103). The collation unit 704 attaches the collation results 101 to 104 as the annotations 305 (step S1104), and updates the non-learning target information 306 based on the collation results 101 to 104 (step S1105). Specifically, for example, the collation unit 704 updates the non-learning target information 306 to “1” for the sample data of the collation results 101 to 104. Then, the output unit 705 outputs the collation results 101 to 104 in the displayable manner (step S1106).

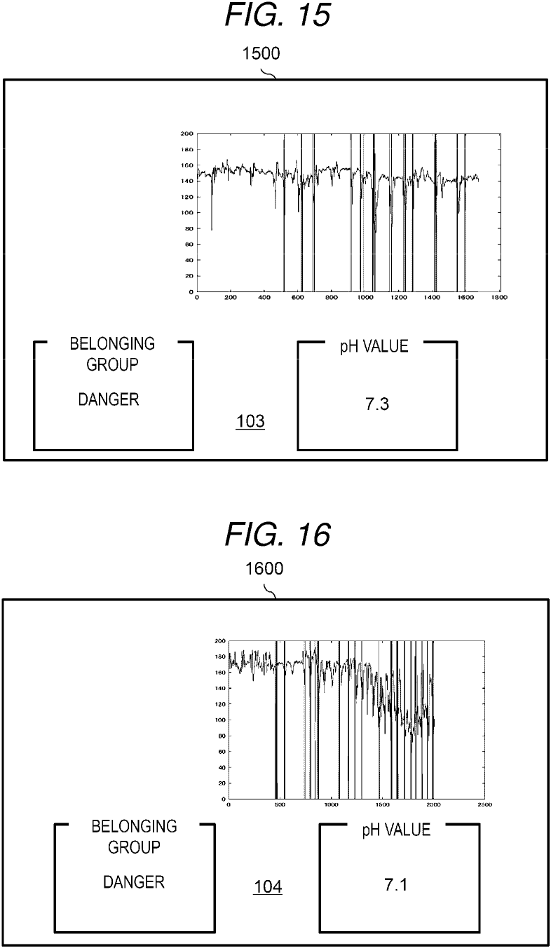

FIG. 13 to FIG. 16 are explanatory diagrams showing examples of collation result display screens. A collation result display screen 1300 displays the collation result 101, a collation result display screen 1400 displays the collation result 102, a collation result display screen 1500 displays the collation result 103, and a collation result display screen 1600 displays the collation result 104.

Returning to FIG. 11, the learning unit 706 executes learning using the learning data set which is a combination of the selection factors of the sample data group and the actual measurement values 332 of the pHs 303 in the collation result 101, and generates the learning model (for example, the multiple regression model) (step S1107). Accordingly, the collation process during learning executed by the grouping processing unit 702, the collation unit 704, the output unit 705, and the learning unit 706 ends.

### Example of Collation Processing Procedure During Prediction

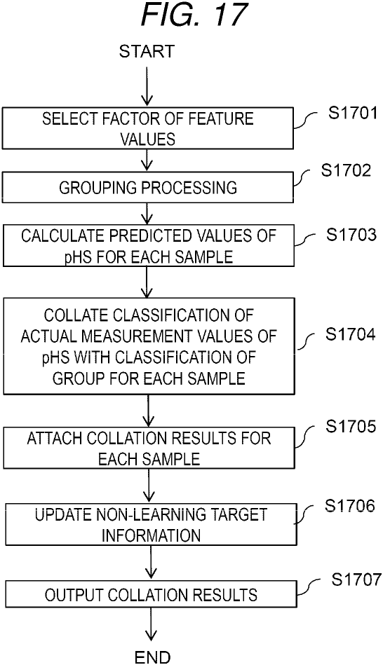

FIG. 17 is a flowchart showing an example of a collation processing procedure during prediction. The grouping processing unit 702 selects a factor of the feature data, as in step S1101 (step S1701). The grouping processing unit 702 executes a grouping process on the sample data group in the learning DB 100 using the factor selected in step S1701 as the feature data 302 (step S1702).

Sample data to be subjected to the grouping process (step S1702) is sample data having no actual measurement values 332 of the pHs 303 and having the non-learning target information 306 of “0” (learning target). Accordingly, since defective data is not applied to the grouping process (step S1702), accuracy of the grouping process (step S1702) during prediction is improved.

During learning, in the grouping process (step S1702), the grouping processing unit 702 may execute the unsupervised learning using only the selection factors of the sample data, or may execute the supervised learning using the selection factors of the sample data and the correct data as the learning data set.

In the case of the supervised learning, the correct data is, for example, a classification (a correct label with both safety as “safety” and a correct label with contradiction and both danger as “danger”) of the annotations 305 attached in step S1104. In any case, the grouping processing unit 702 groups the sample data group into the safety group and the danger group, and sets the belonging groups 304. By executing the supervised learning using the correct data, the collation result 101 attached as the annotations 305 in step S1104 can be reflected in the learning model.

Next, the calculation unit 703 calculates the predicted values 331 of the pHs 303 of the sample data by inputting values of the selection factors of the sample data to the learning model, and stores the predicted values 331 in the learning DB (step S1703) .

Next, as shown in FIG. 1, the collation unit 704 collates a classification (safety or danger) of the predicted values 331 of the pHs 303 with a classification (safety or danger) of the belonging groups 304 for each piece of sample data (step S1704). The collation unit 704 attaches the collation results 101 to 104 as the annotations 305 (step S1705), and updates the non-learning target information 306 based on the collation results 101 to 104 (step S1706).

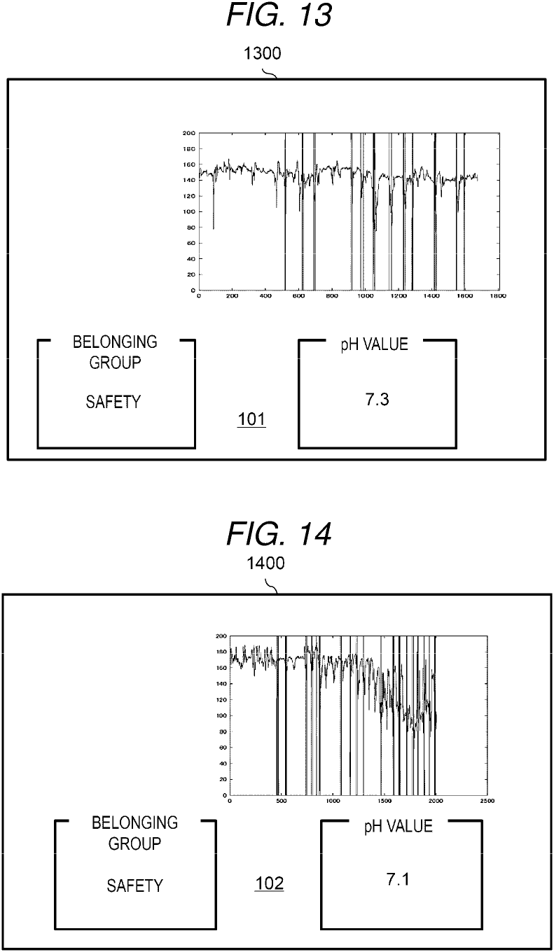

Specifically, for example, the collation unit 704 updates the non-learning target information 306 to “1” for the sample data of the collation results 101 to 104. Then, the collation unit 704 outputs the collation results 101 to 104 in the displayable manner as shown in FIG. 14 to FIG. 17 (step S1707). Accordingly, the collation process during prediction executed by the grouping processing unit 702, the calculation unit 703, the collation unit 704, and the output unit 705 ends.

It is noted that even in a sample having no actual measurement values 332 of the pHs 303 before delivery, the actual measurement values 332 of the pHs 303 are obtained after delivery. In this case, the collation apparatus can update the annotations 305 by executing the collation process during learning shown in FIG. 11 on the actual measurement values 332 of the pHs 303. In addition, the collation apparatus re-learns the learning model by performing error back-propagation based on differences between the predicted values 331 and the actual measurement values 332 of the pHs 303. Accordingly, accuracy of the learning model can be improved.

As described above, since the collation results 101 to 104 indicating “safety”, “contradiction”, and “danger” are automatically attached as the annotations 305, reliability of the annotations 305 can be improved. In addition, since the sample data to which the collation results 102 to 104 indicating “contradiction” or “danger” are attached as the annotations 305 serves as the non-learning target, the sample data to which the collation result 101 indicating “safety” is attached as the annotation 305 remains. Therefore, the automatic organization of the learning DB 100 can be implemented. In addition, by generating the learning model using the remaining sample data group, accuracy of the predicted values 331 of the pHs 303 can be improved. Therefore, it is possible to predict a fetal state without increasing data processing cost.

In the above-described embodiment, two groups of safety and danger are used. However, a classification of groups is not limited to safety and danger, and may be a classification of evaluation indicating a degree such as accuracy or reliability of a sample. In addition, in the above-described embodiment, the sample is a pregnant woman. However, the sample is not limited to a pregnant woman, and various measurement targets such as a person and an apparatus may be used as the sample.

The invention is not limited to the above embodiment and includes various modifications and equivalent configurations within the spirit of the appended claims. For example, the above-mentioned embodiment is described in detail in order to make the invention easy to understand, and the invention is not necessarily limited to those including all the configurations described above. In addition, a part of the configurations according to one embodiment may be replaced with configurations according to another embodiment. In addition, the configurations according to one embodiment may be added to the configurations according to another embodiment. Furthermore, a part of the configurations according to each embodiment may be added to, deleted from, or replaced with another configuration.

A part or all of the configurations, functions, processing units, processing methods described above and the like may be implemented by hardware by, for example, designing with an integrated circuit, or may be implemented by software by a processor interpreting and executing a program for implementing each function.

Information of a program, a table, and a file for implementing each function can be stored in a storage apparatus such as a memory, a hard disk, and a solid state drive (SSD), or a recording medium such as an integrated circuit (IC) card, an SD card, and a digital versatile disc (DVD) .

Control lines and information lines indicate what is considered necessary for description, and not all control lines and information lines necessary for implementation are necessarily shown. It may be considered that almost all the configurations are actually connected to each other.

What is claimed is:

1. A collation apparatus comprising:
a grouping processing unit configured to group, based on each explanatory variable of a sample group, the sample group into a first group indicating a first classification and a second group indicating a second classification having a lower evaluation than the first classification;
a collation unit configured to collate the classification of the first group and the second group that are obtained by the grouping processing unit with a classification identified by each objective variable of the sample group; and
an output unit configured to output a collation result obtained by the collation unit.

2. The collation apparatus according to claim 1, wherein
the collation unit attaches an annotation indicating the first classification to the first sample when the classification identified by the objective variable of the first sample belonging to the first group in the sample group is the first classification.

3. The collation apparatus according to claim 1, wherein
the collation unit attaches an annotation indicating a contradiction to the first sample when the classification identified by the objective variable of the first sample belonging to the first group in the sample group is the second classification.

4. The collation apparatus according to claim 1, wherein
the collation unit attaches an annotation indicating the second classification to the second sample when the classification identified by the objective variable of the second sample belonging to the second group in the sample group is the second classification.

5. The collation apparatus according to claim 1, wherein
the collation unit attaches an annotation indicating a contradiction to the second sample when the classification identified by the objective variable of the second sample belonging to the second group in the sample group is the first classification.

6. The collation apparatus according to claim 1, wherein
the grouping processing unit groups the sample group into the first group and the second group by performing clustering by unsupervised learning using the explanatory variable.

7. The collation apparatus according to claim 1, wherein
the grouping processing unit groups the sample group into the first group and the second group based on supervised learning according to the explanatory variable and a classification identified by an actual measurement value of the objective variable.

8. The collation apparatus according to claim 2, further comprising:
a learning unit configured to generate a learning model by learning based on the explanatory variable of the first sample to which an annotation indicating the first classification is attached and an annotation indicating the first classification serving as the objective variable; and
a calculation unit configured to calculate a predicted value of each objective variable of a prediction target sample group by inputting each explanatory variable of the prediction target sample group to the learning model generated by the learning unit.

9. The collation apparatus according to claim 8, wherein
the grouping processing unit groups the prediction target sample group into the first group and the second group based on each explanatory variable of the prediction target sample group, and
the collation unit collates the classification of the first group and the second group into which the prediction target sample group is grouped by the grouping processing unit with a classification identified by the predicted value calculated by the calculation unit.

10. The collation apparatus according to claim 9, wherein
when a classification identified by the predicted value of a first prediction target sample belonging to the first group in the prediction target sample group is the first classification, the collation unit attaches the annotation indicating the first classification to the first prediction target sample.

11. The collation apparatus according to claim 9, wherein
when a classification identified by the predicted value of a first prediction target sample belonging to the first group in the prediction target sample group is the second classification, the collation unit attaches an annotation indicating a contradiction to the first prediction target sample.

12. The collation apparatus according to claim 9, wherein
when a classification identified by the predicted value of a second prediction target sample belonging to the second group in the prediction target sample group is the second classification, the collation unit attaches an annotation indicating the second classification to the second prediction target sample.

13. The collation apparatus according to claim 9, wherein
when a classification identified by the predicted value of a second prediction target sample belonging to the second group in the prediction target sample group is the first classification, the collation unit attaches an annotation indicating a contradiction to the second prediction target sample.

14. A collation method executed by a collation apparatus, the collation apparatus including a processor configured to execute a program and a storage device configured to store the program, the collation method comprising:
a grouping process of grouping, by the processor, based on each explanatory variable of a sample group, the sample group into a first group indicating a first classification and a second group indicating a second classification having a lower evaluation than the first classification;
a collation process of collating, by the processor, the classification of the first group and the second group that are obtained by the grouping process with a classification identified by each objective variable of the sample group; and
an output process of outputting, by the processor, a collation result obtained by the collation process.

15. A collation program for causing a processor to execute:
a grouping process of grouping, based on each explanatory variable of a sample group, the sample group into a first group indicating a first classification and a second group indicating a second classification having a lower evaluation than the first classification;
a collation process of collating the classification of the first group and the second group that are obtained by the grouping process with a classification identified by each objective variable of the sample group; and
an output process of outputting a collation result obtained by the collation process.
